(19) United States
(12) Patent Application Publication
(10) Pub. No.: US 2016/0310690 A1
(43) Pub. Date: Oct. 27, 2016

(54) COUPLER FOR SAFE DELIVERY OF ANESTHESIA GASES

(71) Applicant: Veranos, Inc., St. Paul, MN (US)

(72) Inventors: Aaron F. Summers, St. Paul, MN (US); Adam D. Codner, St. Paul, MN (US); Brian J. Bosch, Plymouth, MN (US); Jacob G. Fenske, Minneapolis, MN (US); Sean V. Meehan, Minneapolis, MN (US); Timothy P. Ernster, St. Paul, MN (US)

(21) Appl. No.: 14/695,486

(22) Filed: Apr. 24, 2015

(51) Int. Cl. A61M 16/10; A61M 16/08; A61M 16/20; A61M 16/06

(52) U.S. Cl. CPC A61M 16/104; A61M 16/06; A61M 16/0816; A61M 16/0891; A61M 16/201; A61M 2202/0241

(57) ABSTRACT

A breathing circuit delivers anesthetic gases to a patient. The breathing circuit includes a hose having a proximal end coupled to an anesthetic gas delivery machine and a distal end, a breathing apparatus attached to a patient to facilitate patient inhalation and a coupler having a proximal end connected to the distal end of the hose and a distal end connected to the breathing apparatus. The coupler includes a body having an inner surface and an outer surface and a valve that allows gas from the anesthetic gas delivery machine to flow to the breathing apparatus in an opened position and to block gas from flowing from the anesthetic gas delivery machine to the breathing apparatus in a closed position.

### BACKGROUND

The use of general anesthesia includes the delivery of anesthetics to a patient through an anesthesia delivery system. One such way to deliver anesthetics is as a gas through a breathing machine or breathing circuit, which is inhaled by a patient. Volatile anesthetic gases, such as nitrous oxide, sevoflurane, isoflurane and desflurane, can be used to induce general anesthesia or to maintain general anesthesia.

When anesthetic gasses are used to induce general anesthesia, they are used in patients that do not have or are unable to sit still to safely obtain intravenous access and are administered to patients via a mask. The mask is connected to the breathing circuit on the breathing machine and the mask is placed over the patient's mouth and nose. When the patient is fully under general anesthesia via the mask, intravenous access is obtained and intravenous medications are given to facilitate placement of an airway device, such as a laryngeal airway mask, an endotracheal tube or a tracheostomy tube, into the patient. After the airway device is placed in the patient, the breathing circuit is removed from the mask and is attached to the airway device.

When general anesthesia is induced in patients who have intravenous access, an airway device is placed into the patient once the patient is under general anesthesia via intravenous medications. The breathing circuit is then attached to the airway device to maintain general anesthesia.

The discussion above is merely provided for general background information and is not intended to be used as an aid in determining the scope of the claimed subject matter.

### SUMMARY

A breathing circuit that delivers anesthetic gases to a patient. The breathing circuit includes a hose having a proximal end coupled to an anesthetic gas delivery machine and a distal end, a breathing apparatus attached to a patient to facilitate patient inhalation and a coupler having a proximal end connected to the distal end of the hose and a distal end connected to the breathing apparatus. The coupler includes a body having an inner surface and an outer surface and a valve that allows gas from the anesthetic gas delivery machine to flow to the breathing apparatus in an opened position and to block gas from flowing from the anesthetic gas delivery machine to the breathing apparatus in a closed position.

A coupler couples a hose from an anesthetic gas delivery machine to a breathing apparatus attached to a patient and includes a body having an outer surface and an inner surface, a proximal region that couples to a hose from an anesthetic gas delivery machine, a distal region that couples to the breathing apparatus attached to the patient, a medial region located between the proximal region and the distal region and a valve housed within the inner surface of the body of the coupler. In an opened position, the valve allows anesthetic gas to flow from the proximal region to the distal region and wherein in a closed position the valve blocks anesthetic gas from flowing from the proximal region to the distal region and from leaking into a surrounding environment.

A coupler couples a hose from an anesthetic gas delivery machine to a breathing apparatus attached to a patient and includes a body having an outer surface and an inner surface, a proximal region that couples to a hose from an anesthetic gas delivery machine, a distal region that couples to the breathing apparatus attached to the patient, a medial region located between the proximal region and the distal region and a valve accessible from the outer surface of the body of the coupler and manually actuated. In an opened position, the valve allows anesthetic gas to flow from the proximal region to the distal region and wherein in a closed position the valve blocks anesthetic gas from flowing from the proximal region to the distal region and from leaking into a surrounding environment.

A method of preventing anesthetic gases being delivered to a patient by an anesthetic gas delivery machine from entering a surrounding environment includes attaching a proximal end of a coupler to a distal end of a hose that is coupled to an anesthetic gas delivery machine. The coupler includes a valve that in a closed position. The anesthetic gas delivery machine is turned on so that anesthetic gases flow from the anesthetic gas delivery machine, through the hose and to the coupler. The closed valve prevents anesthetic gas from entering into a surrounding environment. A distal end of the coupler is attached to a breathing apparatus that is attached to a patient. Attaching the distal end of the coupler to the breathing apparatus opens the valve so that anesthetic gas flows to the patient.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the background.

### DETAILED DESCRIPTION OF DRAWINGS

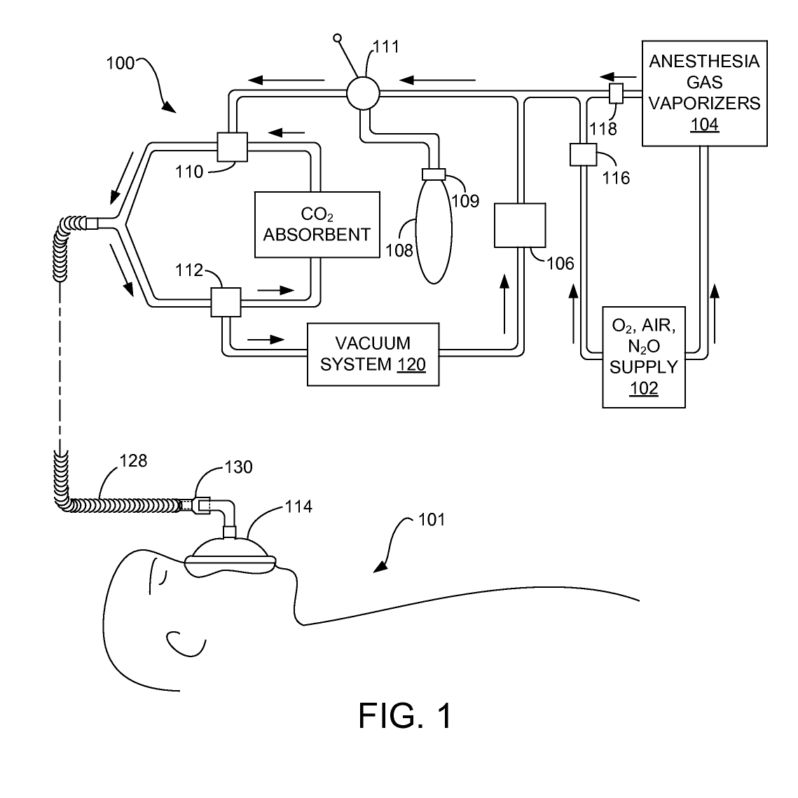

FIG. 1 illustrates a schematic diagram of an anesthesia gas delivery system.

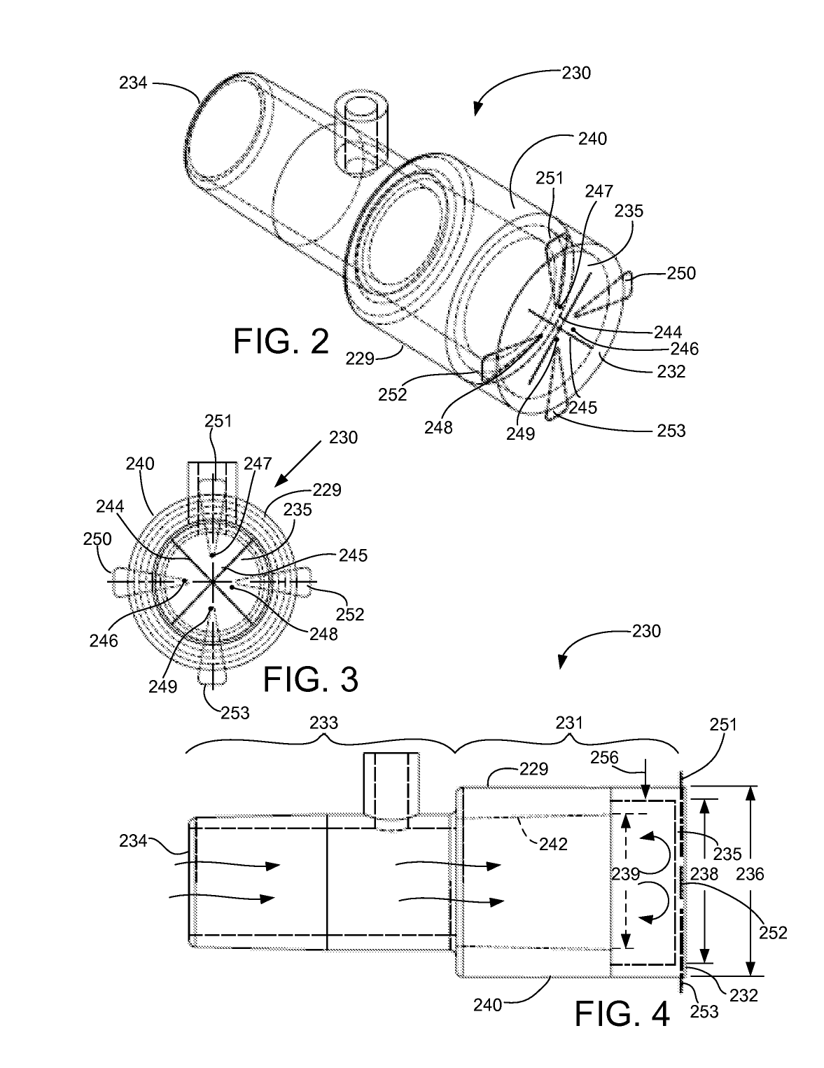

FIG. 2 illustrates a perspective view of a coupler in accordance with one embodiment.

FIG. 3 illustrates an end view of the coupler illustrated in FIG. 2.

FIG. 4 illustrates a side view of the coupler illustrated in FIG. 2.

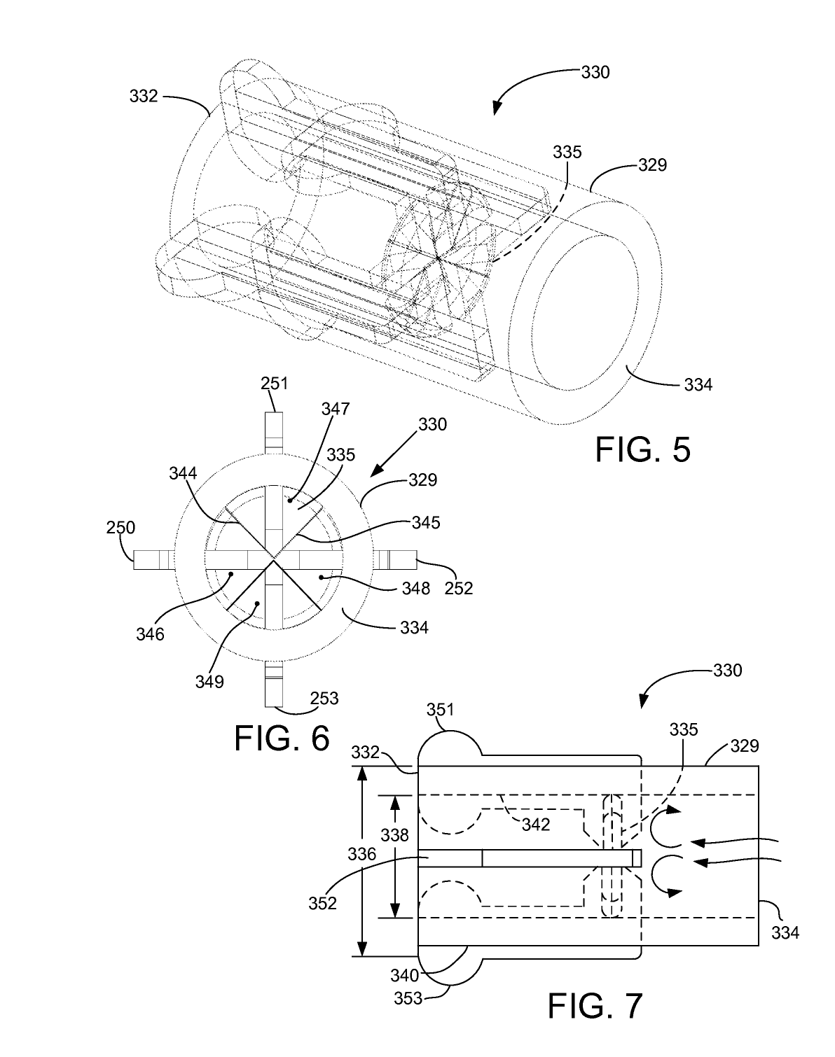

FIG. 5 illustrates a perspective view of a coupler in accordance with another embodiment.

FIG. 6 illustrates an end view of the coupler in FIG. 5.

FIG. 7 illustrates a side view of the coupler in FIG. 5.

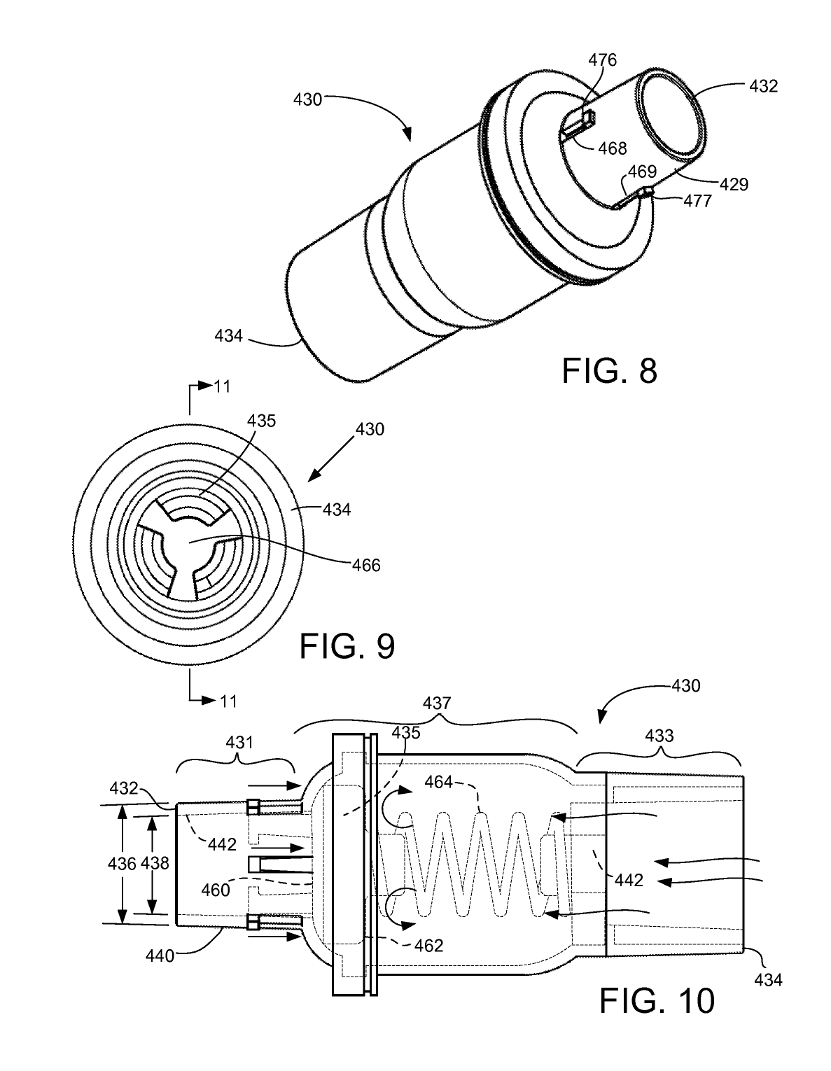

FIG. 8 illustrates a perspective view of a coupler in a closed position in accordance with yet another embodiment.

FIG. 9 illustrates an end view of the coupler in FIG. 8 in the closed position.

FIG. 10 is a side view of the coupler in FIG. 8 in the closed position.

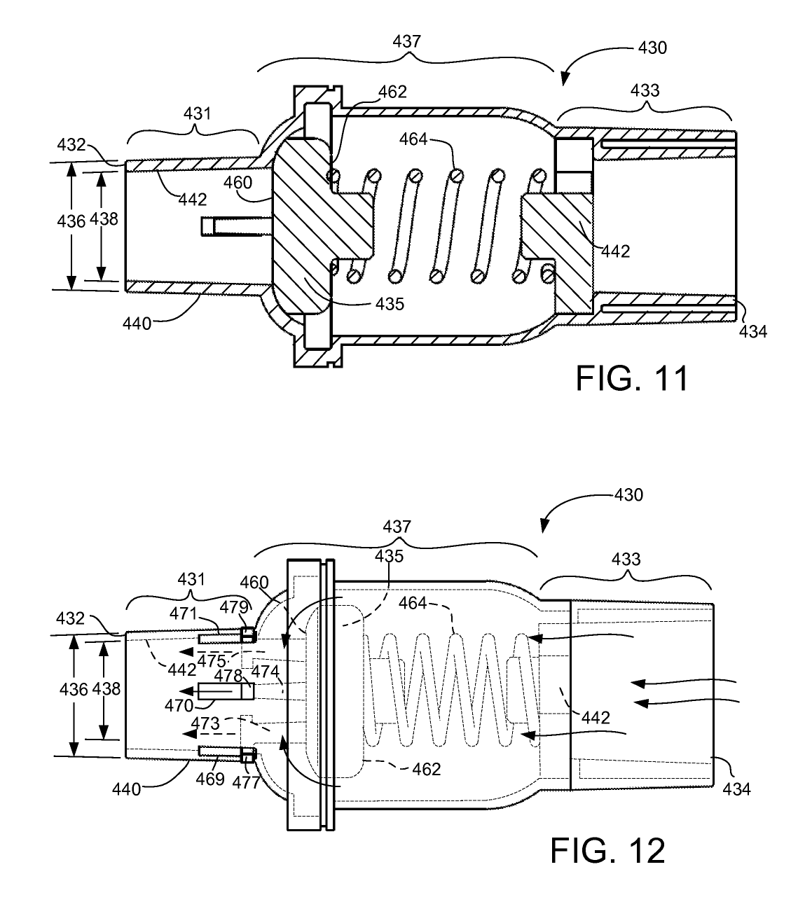

FIG. 11 is a section view of the coupler in FIG. 8 in the closed position taken through the line indicated in FIG. 9.

FIG. 12 illustrates a side view of the coupler in FIG. 8, but in an opened position.

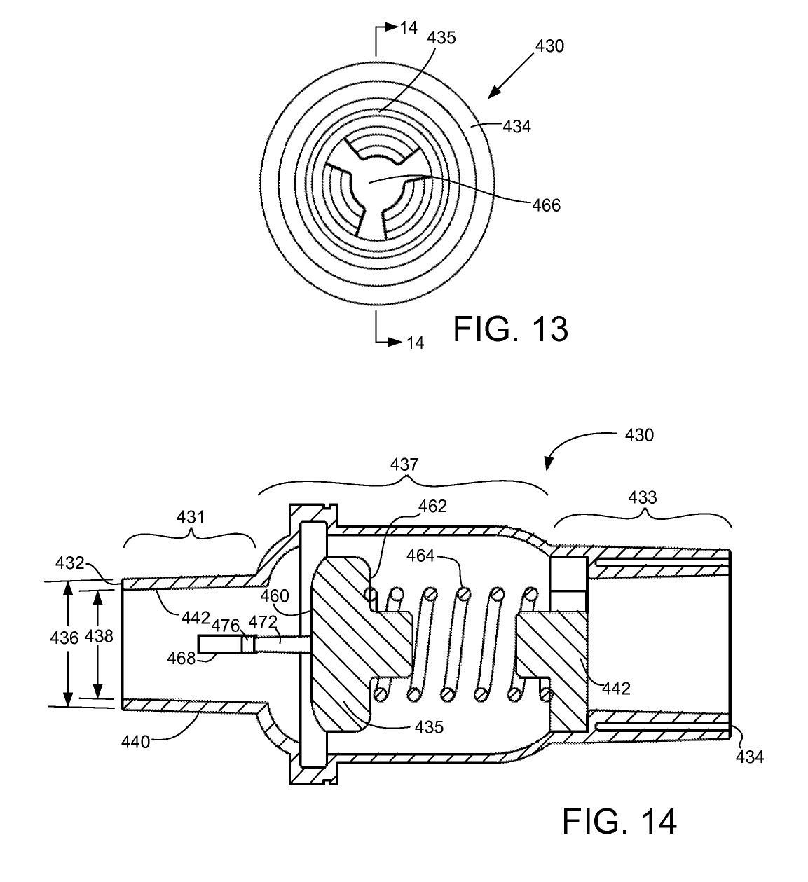

FIG. 13 illustrates an end view of the coupler in FIG. 12 in the opened position.

FIG. 14 illustrates a section view of the coupler in FIG. 12 in the opened position taken through the line indicated in FIG. 13.

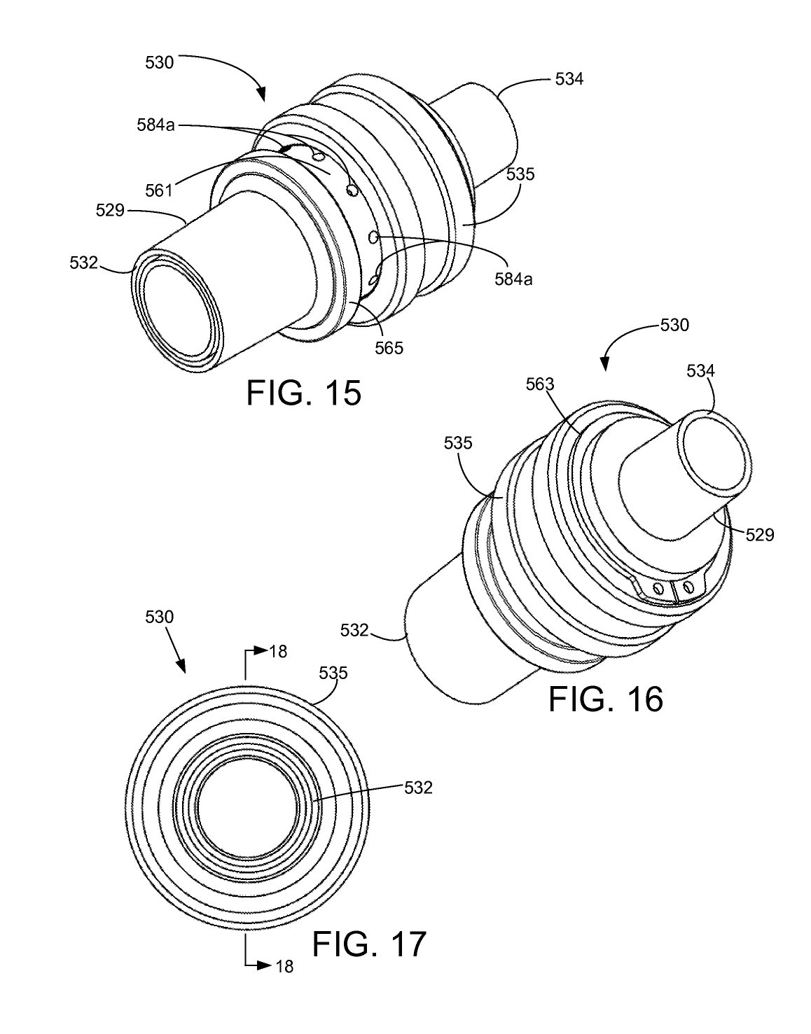

FIG. 15 illustrates a first perspective view of a coupler in a closed position in accordance with still another embodiment.

FIG. 16 illustrates a second perspective view of the coupler in FIG. 15 in the closed position.

FIG. 17 illustrates an end view of the coupler in FIG. 15 in the closed position.

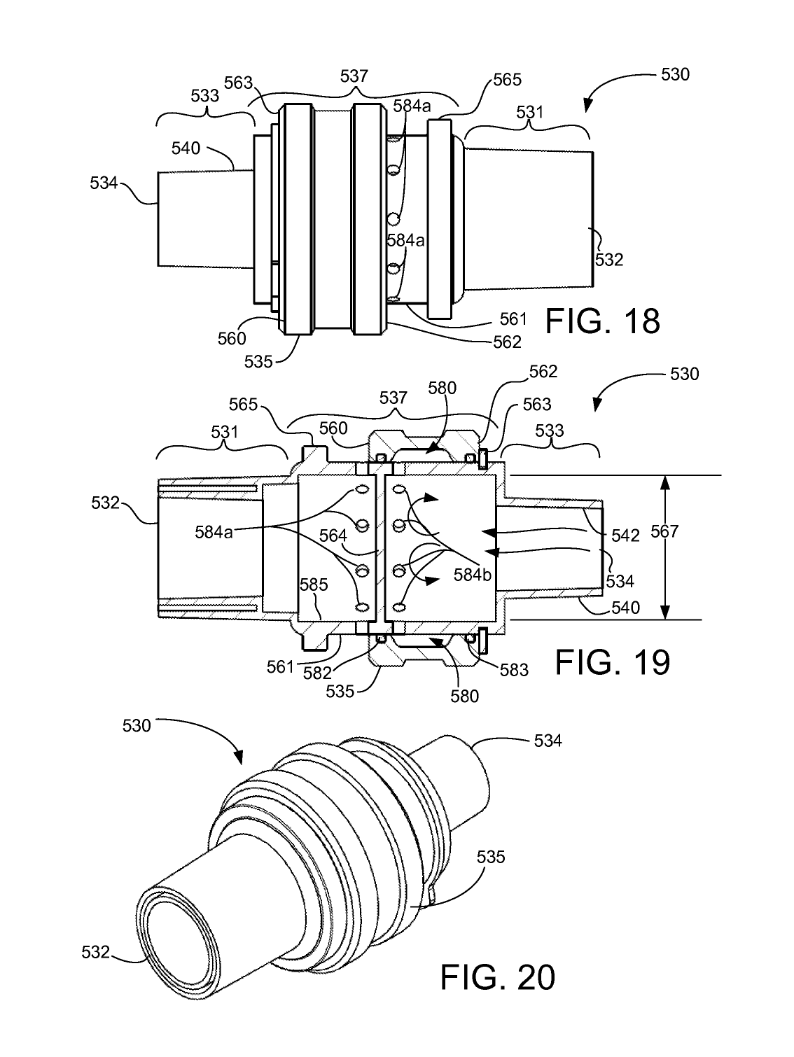

FIG. 18 illustrates a side view of the coupler in FIG. 15 in the closed position.

FIG. 19 illustrates a section view of the coupler in FIG. 15 in the closed position taken through the line indicated in FIG. 17.

FIG. 20 illustrates a first perspective view of the coupler in FIG. 15, but in an opened position.

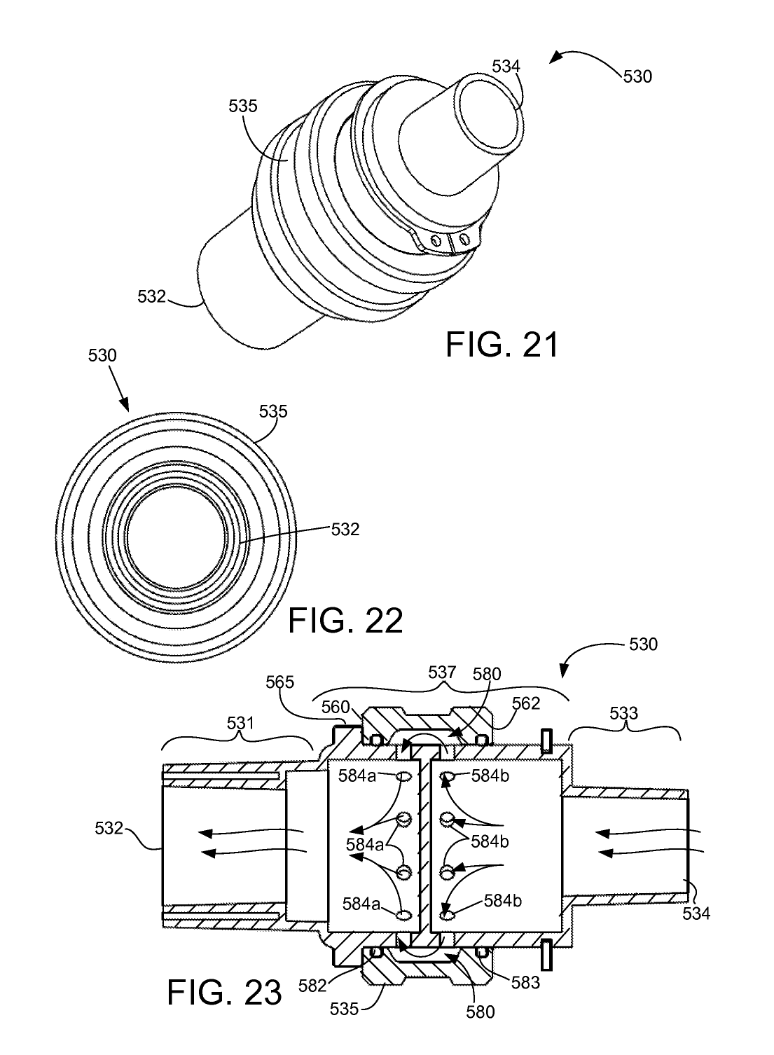

FIG. 21 illustrates a second perspective view of the coupler in FIG. 20 in the opened position.

FIG. 22 illustrates an end view of the coupler in FIG. 20 in the opened position.

FIG. 23 illustrates a section view of the coupler in FIG. 20 in the opened position taken through the line indicated in FIG. 22.

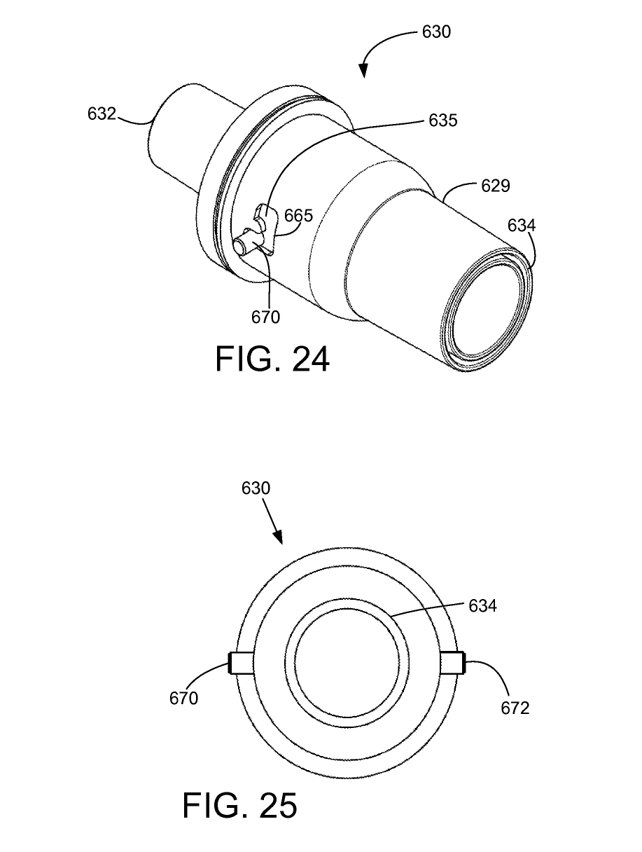

FIG. 24 illustrates a perspective view of a coupler in accordance with still another embodiment.

FIG. 25 illustrates an end view of the coupler illustrated in FIG. 24.

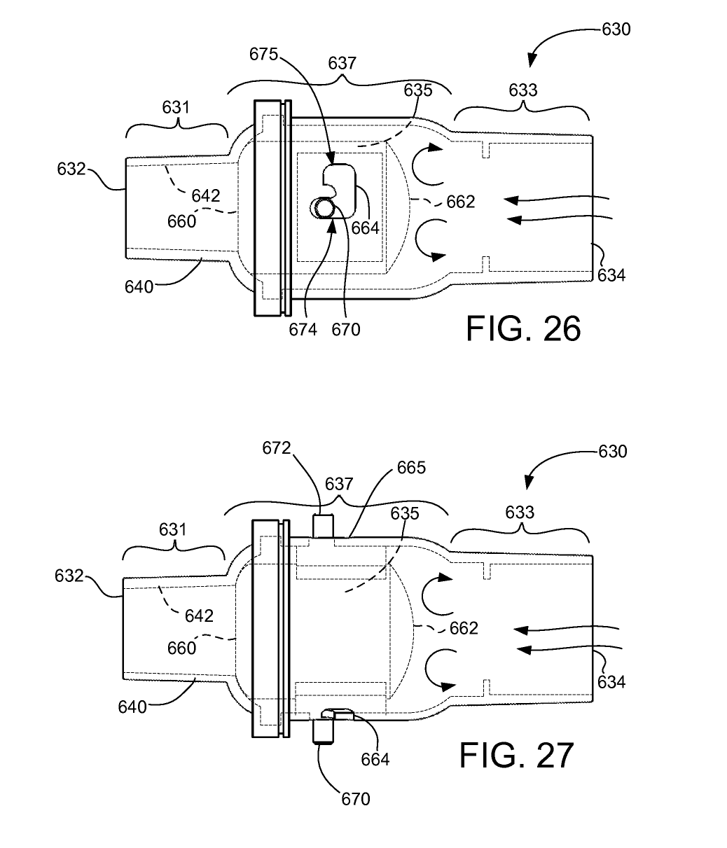

FIG. 26 illustrates a side view of the coupler illustrated in FIG. 24.

FIG. 27 illustrates a top view of the coupler illustrated in FIG. 24.

### DETAILED DESCRIPTION

FIG. 1 illustrates a schematic diagram of an anesthesia gas delivery system or machine 100. When anesthesia gas delivery system 100 is coupled to a patient 101, system or machine 100 forms a breathing circuit. The breathing circuit includes an oxygen, air, N₂O gas supply 102, anesthesia gas vaporizers 104, a ventilator piston unit 106, a reservoir bag 108 with corresponding APL (adjusting pressure limiting) valve 109, an inspiration valve 110, an expiration valve 112, and a breathing apparatus 114. Breathing apparatus 114 attaches to patient 101 to facilitate patient inhalation. Example types of breathing apparatuses includes a mask as illustrated or other airway devices, such as a tracheal or breathing tubes. Anesthetic machine 102 is a device for supplying anesthetic gases. In general, anesthetic machine 102 includes the basic function of receiving compressed gases from supplies and creating a gas mixture of known composition and flow rate at a common gas outlet (CGO) to breathing circuit 104. Anesthetic machine 102 also includes safety features designed to prevent the delivery of a hypoxic mixture to breathing circuit 104.

Gas supply 102 supplies anesthesia delivery machine 100 with a supply of oxygen, air, and nitrous oxide (N₂O) and partially flows to anesthesia gas vaporizers 104, which stores volatile anesthetics (i.e. isoflurane, desflurane, sevofluran, etc.) and partially flows to an oxygen flush valve 116. The split of gases 102 reconnect and a check valve 118 prevents backward flow into vaporizers 104. After the gases 102 pass through vaporizers 104, the gases 102 can flow directly to the patient or can be driven there. Gases 102 are automatically driven by ventilator piston unit 106 or manually driven by an anesthesia provider via reservoir bag 108 depending on bag/vent selector switch 111. The pressure of reservoir bag 108 is adjusted via APL valve 109. Inspiration valve 110 is opened and expiration valve 112 is closed during inspiration and inspiration valve 110 is closed and expiration valve 112 is opened during expiration. Expired gases are scavenged via a vacuum system 120.

When a patient, such as patient 101, is under general anesthesia, there can be frequent disconnections and reconnections of a breathing apparatus, such as mask 114 or a breathing tube, from anesthesia gas delivery machine 100 causing entrainment of anesthetic gases into the operating room environment. For example, when patient 101 is placed under general anesthesia by inhaling anesthetic gases through mask 114, eventually mask 114 is removed from the patient to insert an airway device. Mask 114 is then removed from an end of the breathing circuit of anesthesia gas delivery machine 100 and attached to the airway device. In another example, when a patient is intravenously placed under general anesthesia, anesthetic gases are turned on, often prior to airway placement and connection to anesthesia gas delivery machine 100. In addition, during the course of general anesthesia, the patient may need to be positioned, repositioned or turned (i.e., supine to prone). As a matter of course, the anesthetic breathing circuit is disconnected from the airway device. This is done as a safety precaution to avoid dislodging the airway device while moving the patient.

The entrainment of anesthetic gases into the operating room environment not only exposes healthcare staff to unnecessary anesthetic gases, but also contributes to excess expenditure of these gases and potential environmental pollution. A coupler 130 is described herein that couples to an end of a breathing circuit on one end (via hose 128) and couples to a breathing apparatus, such as a mask 114 or airway device, on its opposing end. Coupler 130 is made of polyvinyl chloride free plastic and is disposable. Coupler 130 includes a valve that prevents the escape of anesthetic gases into the operating room during breathing circuit disconnection from a patient's breathing apparatus, provides safe delivery of anesthetic gases and provides a more efficient way of preventing environmental pollution in the operating room and allows for greater conservation and costs savings of anesthetic gases.

In one embodiment, coupler 130 is an automatic device. An automatic coupler includes a valve that automatically closes to stop the flow of anesthetic from the breathing circuit into the environment when the breathing apparatus is removed or detached from the coupler. When the mask or airway device is reconnected, the valve automatically opens to allow anesthetic gas from machine 100 to flow to the patient. In another embodiment, coupler 130 is a manual device. A manual coupler allows for more flexibility to an anesthesia provider given the multitude of ways anesthesia providers practice. In the automatic versions, the flow of anesthesia gases is stopped only when the breathing apparatus is disconnected from coupler 130. Some providers, in the case where a patient is wearing a mask and does not yet have an airway, likes to leave the mask connected to the breathing circuit in case the patient might need to receive mask ventilation quickly. With the manual version of coupler 130 and prior to the mask or airway device being disconnected from the breathing circuit, coupler 130 includes a valve that is manually closed to stop the flow of anesthetic gas from the breathing circuit to the patient. The breathing apparatus and breathing circuit can then be removed from the patient. After reattaching the breathing apparatus and the breathing circuit to the patient, the valve is manually opened to allow anesthetic gas to flow to the patient.

FIG. 2 illustrates a perspective view of a coupler 230 in accordance with one embodiment. FIG. 2 is illustrated with broken interior phantom lines. FIG. 3 illustrates an end view of coupler 230 with broken interior phantom lines and FIG. 4 illustrates a side view of coupler 230 with broken interior phantom lines. Coupler 230 is an exemplary embodiment of a coupler having an automatic valve that could be used in place of coupler 130 illustrated in FIG. 1. Coupler 230 is automatically opened when connected to a mask or airway device. Coupler 230 includes a body 229, a distal end 232, a proximal end 234 and a valve 235. Further and as illustrated in FIG. 4, coupler includes a distal region 231 and a proximal region 233. Distal region 231 is coupleable to a mask or airway device and proximal region 233 is coupleable to a hose, such as hose 128, for attachment to an anesthesia gas delivery machine, such as machine 100. Therefore, gas from an anesthesia gas delivery system flows from proximal end 234 to distal end 232 in coupler 230. FIG. 3 illustrates a proximal end view of coupler 230. In FIGS. 2-4, valve 235 is in a closed position because neither a mask, such as mask 114, nor an airway device, such as a breathing tube, is coupled to distal region 231 of coupler 230. As illustrated by the arrows in FIG. 4, anesthetic gasses stop a valve 235 so as not to flow to a breathing apparatus and not leak into the surrounding environment.

Distal region 231 of coupler 230 includes a standard fitting for coupling to a mask or for coupling to an airway device like a breathing or tracheal tube. More specifically and as best illustrated in FIG. 4, distal region 231 includes an outer diameter 236, a first inner diameter 238 and a second inner diameter 239 that may or may not taper. According to standard sizing, outer diameter 236 is dimensioned such that outer surface 240 of body 229 of distal region 231 receives a portion of a mask for connecting to and sealing to the mask, while second inner diameter 239 is dimensioned such that inner surface 242 of body 229 of distal region 231 receives a portion of an airway device, such as a breathing tube, for connecting to and sealing to the airway device.

As best illustrated in FIGS. 2 and 3, valve 235 extends across first inner diameter 238 of distal region 231, is flexible and includes a pair of intersecting slits 244 and 245 that form four flexible and triangular flaps 246, 247, 248 and 249. Each flap 246, 247, 248 and 249 includes a corresponding leaflet 250, 251, 252 and 253, respectively. Leaflets 250-253 are flat and triangular in shape. More specifically, leaflets are flat and in the shape of an isosceles triangle with a base of each leaflet being located a distance from outer surface 240 of distal region 231 and the vertex point of each leaflet being attached to one of the four flexible flaps 246-249. It should be realized that other leaflet shapes are possible. However, one side or end of the leaflet radially extends from outer surface 240 of body 229 of distal region 231 and an opposing side or end of the leaflet is attached to one of the flaps of valve 235.

When a portion of a mask, which seals to outer surface 240 of distal region 231, is connected to coupler 230, the mask pushes the bases of leaflets or the portions of leaflets that are radially extending outwardly from outer surface 240 against outer surface 240 and therefore actuates flexible flaps 246-249 of valve 235 into an opened position. In the opened position, patient 101 can inspire anesthetic gases from anesthesia gas delivery machine 100.

In the alternative, when a portion of an airway device or breathing tube, which seals to second inner surface 242 of distal region 231, is connected to coupler 230, the portion of the airway device or breathing tube that pushes against flexible flaps 246-249 and therefore against the vortex points of leaflets 250-253 that are attached to flexible flaps 246-249. Valve 235 is actuated into an opened position and the flexible flaps 246-249 and attached leaflets 250-253 are folded out of the way of the airway device or breathing tube into the radial space 256 between second inner diameter 242 or the seal surface and first inner diameter 238.

When the mask or airway device is removed, valve 235 returns to a closed position, preventing anesthesia gases from flowing through coupler 230 and therefore preventing exposure of anesthesia gases to the surrounding environment.

FIG. 5 illustrates a perspective view of a coupler 330 in accordance with another embodiment. FIG. 5 is illustrated with broken interior phantom lines. FIG. 6 illustrates an end view of coupler 330 and FIG. 7 illustrates a side view of coupler 330, each view illustrating broken interior phantom lines. Like coupler 230, coupler 330 is an exemplary embodiment of a coupler having an automatic valve that could be used in place of coupler 130 illustrated in FIG. 1. Coupler 330 is automatically opened when connected to a mask or airway device. Coupler 330 includes a body 329, a distal end 332, a proximal end 334 and a valve 335. Gas from an anesthesia gas delivery machine flows from proximal end 334 to distal end 332 in coupler 330. FIG. 6 illustrates a proximal end view of coupler 330. In FIGS. 5-7, valve 335 is in a closed position because neither a mask, such as mask 114, nor an airway device, such as a breathing tube, is coupled to coupler 230. As illustrated by the arrows in FIG. 7, anesthetic gases are stopped at valve 335 so as not to flow to a breathing apparatus and not leak into the surrounding environment.

Distal end 332 of coupler 330 includes a standard fitting for coupling to a mask or for coupling to an airway device like a breathing or tracheal tube. More specifically and as best illustrated in FIG. 7, distal end 332 includes an outer diameter 336 and an inner diameter 338. According to standard sizing, outer diameter 336 is dimensioned such that outer surface 340 of body 329 of distal end 332 receives a portion of a mask for connecting to and sealing to the mask, while inner diameter 338 is dimensioned such that inner surface 342 of body 329 of distal end 332 receives a portion of an airway device, such as a breathing tube, for connecting to and sealing to the airway device.

As illustrated, valve 335 extends across inner diameter 338 of coupler 330, is flexible and includes a pair of intersecting slits 344 and 345 that form four flexible and triangular flaps 346, 347, 348 and 349. Attached to each flap 346, 347, 348 and 349 is a first end of a moveable leg 350, 351, 352 and 353, respectively. Bulbous second ends of moveable legs 350-353 and other sections of moveable legs 350-353 partially protrude outwardly from outer surface 340 of body 329 and are partially located internal to inner surface 342 of body 329. It should be realized that other moveable leg shapes are possible. However, one end of each moveable leg must be located a distance from outer surface 340 of body 329 and an opposing end of each moveable leg must be attached to one of the flaps of valve 335.

When a portion of a mask, which seals to outer surface 340 of body 329 is connected to coupler 330 the mask pushes the bulbous ends of each moveable leg 350-253 or the portion of each moveable leg that is extending outwardly from outer surface 340 of body 329 so that the entirety of the bulbous ends are located inwardly from inner surface 342 and therefore actuates flexible flaps 346-349 of valve 335 into an opened position. In the opened position, patient 101 can inspire anesthetic gases from anesthesia gas delivery system 100.

In the alternative, when a portion of an airway device or breathing tube, which seals to inner surface 342 of body 329 is connected to coupler 330, the portion of the airway device or breathing tube pushes the bulbous ends of each moveable leg 350-353 or the portion of each moveable leg that is extending inwardly from inner surface 342 so that the entirety of the bulbous ends are located outwardly from outer surface 340 and therefore actuated flexible flaps 346-349 of valve 335 into an opened position.

When the mask or airway device is removed, valve 335 returns to a closed position as illustrated in FIGS. 5-7, preventing anesthesia gases from flowing through coupler 330 and therefore preventing exposure of anesthesia gases to the surrounding environment.

FIG. 8 illustrates a perspective view of a coupler 430 in a closed position in accordance with yet another embodiment. FIG. 9 illustrates an end view of coupler 430 in the closed position, FIG. 10 is a side view of coupler 430 in the closed position with broken interior phantom lines and FIG. 11 is a section view of coupler 430 in the closed position taken through the line indicated in FIG. 9. Like couplers 230 and 330, coupler 430 is an exemplary embodiment of a coupler having an automatic valve that could be used in place of coupler 130 illustrated in FIG. 1. Coupler 430 can be automatically opened when connected to a mask or airway device. Coupler 430 includes a body 429, a distal end 432, a proximal end 434 and a valve 435. Further and as illustrated in FIGS. 10 and 11, coupler 430 includes a distal region 431 and a proximal region 433. Distal region 431 is coupleable to a mask or airway device and proximal region 433 is coupleable to a hose for attachment to an anesthesia gas delivery machine. Therefore, gas from an anesthesia gas delivery machine flows from proximal end 434 to distal end 432 in coupler 430. FIG. 9 illustrates a proximal end view of coupler 430.

Distal region 431 of coupler 430 includes a standard fitting for coupling to a mask or for coupling to an airway device like a breathing or tracheal tube. More specifically and as best illustrated in FIGS. 10 and 11, distal region 431 includes an outer diameter 436 and an inner diameter 438 that may or may not taper. As illustrated in FIGS. 10 and 11, outer diameter 436 and inner diameter 438 taper. According to standard sizing, outer diameter 436 is dimensioned such that outer surface 440 of body 429 in distal region 431 receives a portion of a mask for connecting to and sealing to the mask, while inner diameter 438 is dimensioned such that inner surface 442 of body 429 in distal region 431 receives a portion of an airway device, such as a breathing tube, for connecting to and sealing to the airway device.

As best illustrated in FIGS. 10 and 11, coupler 430 further includes a medial region 437 located between distal region 431 and proximal region 432. Medial region 437 includes an outer diameter and an inner diameter that are greater than outer diameter 436 and inner diameter 438 of distal region 431. Valve 435 is located in medial region 437 and has a distal end 460 and a proximal end 462. Medial region 437 also includes a spring 464 that is coupled to a stop 466 on one end and proximal end 462 of valve 435 at an opposing end. Stop 466 attaches to the interior surface of a portion of proximal region 433. As illustrated in FIG. 9 and in the section view in FIG. 11, stop 466 does not block the flow of gases in the interior of coupler 430. Rather, stop 466 attaches to the interior surface in some areas and allows gas to pass stop 466 in other areas. However, it should be realized that stop 466 can have other configurations than those illustrated just as long as it provides a mounting area for spring 464 and allows gas to pass from proximal region 433 to distal region 431.

When spring 464 is in a neutral position as is illustrated in FIGS. 10 and 11, spring biases distal end 460 of valve 435 against the curved interior surface of medial region 437 that is connected to the interior surface 442 of distal region 431. Distal end 460 includes a surface having a silicone bond so that it is capable of sealing to interior surface. In this way, valve 435 is closed or valve 435 acts as a plug to block the passage of gas from proximal region 433 and medial region 437 into distal region 431. As illustrated by the arrows in FIG. 10, anesthetic gases are stopped at valve 435 so as not to flow to a breathing apparatus and no leak into the surrounding environment.

FIG. 12 illustrates a side view of coupler 430 in an opened position with broken interior phantom lines. FIG. 13 illustrates an end view of coupler 430 in the opened position and FIG. 14 illustrates a section view of coupler 430 in the opened position taken through the line indicated in FIG. 13. Coupler 430 further includes a plurality of slots 468, 469, 470 and 471 that extend through the wall from outer surface 440 to inner surface 442 of distal region 431 and a plurality of rigid prongs 472, 473, 474 and 475 having proximal ends that are attached to distal end 460 of valve 435 and having distal ends 476, 477, 478 and 479. A portion of distal ends 476, 477, 478 and 479 protrude through respective slots 468, 469, 470 and 471. Although FIGS. 8-14 illustrate coupler 430 having four slots and four corresponding prongs and distal ends, it should be realized that any number of slots and corresponding prongs and distal ends can be used including at least one.

When a portion of a mask, which seals to outer surface 440 of body 429 in distal region 431, is connected to coupler 430, the mask pushes the portion of distal ends 476, 477, 478 and 479 of prongs 472, 473, 474 and 475 that are extending outwardly from outer surface 440 through slots 468, 469, 470 and 471 and thereby biases spring 464 toward stop 442. In this way, distal ends 476, 477, 478 and 479 slide from the position illustrated in FIGS. 8-11 to the opened position illustrated in FIGS. 12-14 as indicated by the arrows in FIG. 10 and proximally moves distal end 460 of valve 435 away from the curved interior surface of medial region 437. In the opened position illustrated in FIGS. 12-14, patient 101 can inspire anesthetic gases from anesthesia gas delivery system 100 and there is no leakage of anesthetic gases to the environment because the mask, when completely connected, fully covers slots 468, 469, 470 and 471 on outer surface 440. Arrows in FIG. 12 illustrated the flow of anesthetic gases.

In the alternative, when a portion of an airway device or breathing tube, which seals to inner surface 442 of distal region 431 is connected to coupler 430, the portion of the airway device or breathing tube pushes the remaining portion of distal ends 476, 477, 478 and 479 of prongs 472, 473, 474 and 475 that are located inside coupler 430 and thereby biases spring 464 toward stop 442. In this way, distal ends 476, 477, 478 and 479 slide from the position illustrated in FIGS. 8-11 to the opened position illustrated in FIGS. 12-14 as indicated by the arrows in FIG. 10. In this alternative use, there is also no leakage of anesthetic gases to the environment because the airway device, when completed connected, fully covers slots 468, 469, 470 and 471 on inner surface 442.

When the mask or airway device is removed, spring 464 returns to its neutral position and therefore valve 435 returns to a closed position, preventing anesthesia gases from flowing through coupler 430 and therefore preventing exposure of anesthesia gases to the surrounding environment. In this way, distal ends 476, 477, 478 and 479 slide from the position illustrated in FIGS. 12-14 to the position illustrated in FIGS. 8-11 as indicated by the arrows in FIG. 12.

FIG. 15 illustrates a first perspective view of a coupler 530 in a closed position in accordance with still another embodiment and FIG. 16 illustrates a second perspective view of coupler 530. FIG. 17 illustrates an end view of coupler 530 in the closed position, FIG. 18 illustrates a side view of coupler 530 in the closed position and FIG. 19 illustrates a section view of coupler 530 in the closed position taken through the line indicated in FIG. 17. Unlike couplers 230, 330 and 430, coupler 530 is an exemplary embodiment of a coupler having a manual valve that could be used in place of coupler 130 illustrated in FIG. 1. Coupler 530 can be manually opened from the outside of coupler 530 after a mask or airway device is connected. Coupler 530 includes a body 529, a distal end 532, a proximal end 534 and a valve 535. Valve 535 is a sleeve that can be slid along an outer surface of coupler 530. Further and as illustrated in FIGS. 18 and 19, coupler 530 includes a distal region 531 and a proximal region 533. Distal region 531 is coupleable to a mask or airway device and proximal region 533 is coupleable to a hose for attachment to an anesthesia gas delivery system. Therefore, gas from an anesthesia gas delivery system flows from proximal end 534 to distal end 532 in coupler 530. FIG. 15 is a distal end perspective view, FIG. 16 is a proximal end perspective view and FIG. 17 is a distal end view of coupler 530.

Distal region 531 of coupler 530 includes a standard fitting for coupling to a mask or for coupling to an airway device like a breathing or tracheal tube. More specifically and as best illustrated in FIGS. 18 and 19, distal region 531 includes an outer diameter and an inner diameter that may or may not taper. According to standard sizing, the outer diameter of distal region 531 is dimensioned such that outer surface 540 of distal region 531 of coupler 530 receives a portion of a mask for connecting to and sealing to the mask, while the inner diameter is dimensioned such that an inner surface 542 of distal region 531 receives a portion of an airway device, such as a breathing tube, for connecting to and sealing to the airway device.

As illustrated, coupler 530 further includes a medial region 537 located between distal region 531 and proximal region 532. Valve 535 is located in medial region 537 and has a distal end 560, a proximal end 562, a channel 580 and a pair of O-rings 582 and 583. More particularly, valve 535 is slidably coupled to an exterior or an outer surface 561 of medial region 537 and bounded between a proximally located snap lock 563 and a distally located protrusion 565. Medial region 537 also includes a partition 564 that spans across the internal diameter 567 of medial region 537 of coupler 530 in its entirety and is located between proximally located snap lock 563 and distally located protrusion 565. Still further, medial region 537 includes a plurality of apertures 584 extending between the inner surface of medial flange 585 defined by inner diameter 567 and outer surface 561 of medial flange 537. A first set of apertures 584a are located distally to partition 564 and a second set of apertures 584b are located proximally to partition 564.

In the closed position illustrated in FIGS. 15-19, valve 535 is slid so that proximal end 562 of valve 530 is located adjacent to proximally located snap lock 563 and held in this position by pair of O-rings 582 and 583. In this position, gas (as indicated by the illustrated arrows in FIG. 19) is prevented from flowing from proximal end 534 to distal end 532 in coupler 530. Valve 535 is positioned such that in combination with wall 564, the gas is unable go anywhere but stay within a portion of medial region 537 and in proximal region 533. O-rings 582 and 583 create air-tight seals so any gas that permeates apertures 584b and into channel 580 is unable to leak to the environment. As illustrated in FIGS. 15, 18 and 19, apertures 584a are uncovered and exposed. However, anesthesia gas is trapped within coupler 530 in the portion of the medial region 537 that is in communication with proximal region 533 by wall 564 as shown by the arrows in FIG. 19.

FIG. 20 illustrates a distal end perspective view of coupler 530 in an opened position and FIG. 21 illustrates a proximal end perspective view of coupler 530 in the opened position. FIG. 22 illustrates a distal end view of coupler in the opened position and FIG. 23 illustrates a section view of coupler 530 in the opened position taken through the line indicated in FIG. 22.

In the opened position illustrated in FIGS. 20-23, valve 535 is slid so that distal end 560 of valve 530 is located adjacent to distally located protrusion 565 and held in this position by pair of O-rings 582 and 583. In this position, gas (as indicated by the illustrated arrows in FIG. 23) flows from proximal end 534 to distal end 532 in coupler 530 by flowing through apertures 584b, through channel 580 and through apertures 584a. O-rings 582 and 583 create air-tight seals so the gas permeating apertures 584b, flowing through channel 580 and permeating apertures 584a is unable to leak to the environment. As illustrated in FIGS. 20 and 23, apertures 584a and 584b are covered.

FIG. 24 illustrates a perspective view of a coupler 630 in accordance with still another embodiment. FIG. 25 illustrates an end view of coupler 630, FIG. 26 illustrates a side view of coupler 630 and FIG. 27 illustrates a top view of coupler 630. Like coupler 530, coupler 630 is an exemplary embodiment of a coupler having a manual valve that could be used in place of coupler 130 illustrated in FIG. 1. Coupler 630 can be manually opened from the outside after a mask or airway device is connected. Coupler 630 includes a body 629, a distal end 632, a proximal end 634 and a valve 635. Further and as illustrated in FIGS. 26 and 27, coupler 630 includes a distal region 631 and a proximal region 633. Distal region 631 is coupleable to a mask or airway device and proximal region 633 is coupleable to a hose for attachment to an anesthesia gas delivery system. Therefore, gas from an anesthesia gas delivery system flows from proximal end 634 to distal end 632 in coupler 630. FIG. 25 illustrates a proximal end view of coupler 630.

Distal region 631 of coupler 630 includes a standard fitting for coupling to a mask or for coupling to an airway device like a breathing or tracheal tube. More specifically and as best illustrated in FIGS. 26 and 27, distal region 631 includes an outer diameter and an inner diameter that may or may not taper. According to standard sizing, the outer diameter of distal region 631 is dimensioned such that outer surface 640 of distal region 631 of coupler 630 receives a portion of a mask for connecting to and sealing to the mask, while the inner diameter is dimensioned such that an inner surface 642 of distal region 631 receives a portion of an airway device, such as a breathing tube, for connecting to and sealing to the airway device.

As illustrated in FIGS. 26 and 27, coupler 630 further includes a medial region 637 located between distal region 631 and proximal region 632. Medial region 637 includes an outer diameter and an inner diameter that are greater than the outer diameter and the inner diameter of distal region 631. Valve 635 is housed within the interior of medial region 637 and has a distal end 660, a proximal end 662 and a pair of handles 670 and 672 located on opposing lateral sides of valve 635. The lateral sides of valve are flush against the sides of medial flange 637 so that there is no leakage of anesthetic gases to the environment. Each handle 670 and 672 protrudes through one of a pair of openings 664 and 665 located on opposing lateral sides of medial region 637 of coupler 630. Openings 664 and 665 extend between an inner surface of medial region 637 and an outer surface of medial region 637 of coupler 630. As illustrated in FIGS. 24, 26 and 27, openings 664 and 665 include a first area 674 (the first area is not shown for opening 665 but is similar to first area 674 of opening 664) for handles 670 and 672 to protrude through and a second area 675 (the second area is not shown for opening 665 but is similar to second area 675 of opening 664) for handles 670 and 672 to protrude through. When handles 670 and 672 are protruding through first area 674 as illustrated in FIGS. 24-27, distal end 660 of valve 635 located inside medial region 637 of coupler 630 is positioned against the curved interior surface of medial region 637 that is continuous with the interior surface 642 of distal region 631. Distal end 660 includes a surface having a silicone bond so that it is capable of sealing to another surface. In this way, valve 635 is closed or valve 635 acts as a plug to block the passage of gas from proximal region 633 and medial region 637 into distal region 631 as is shown by the arrows in FIGS. 26 and 27.

To actuate valve 635 into an opened position, handles 670 and 672 are moved to be located into and protrude through second area 675 of openings 664 and 665. When one of handles 670 and 672 are actuated into this position, the opposing handle is also moved into this position. When handles 670 and 672 are protruding through second area 675, distal end 660 of valve 635 is moved proximally away from the curved interior surface of medial region 637. In the opened position, patient 101 can inspire anesthetic gases from anesthesia gas delivery system 100 and there is no leakage of anesthetic gases to the environment because the lateral sides of valve which include handles 670 and 672 remain flush against the sides of medial region 637 and therefore continue to block openings 662 and 664. To return valve 635 to a closed position, handles 670 and 672 are returned to first area 674 of openings 664 and 665.

In other embodiments, coupler 130 can provide a valve that both automatically opens and closes as well as manually opens and closes. For example, coupler 130 could use the automatic configuration illustrated in FIG. 2-4, 5-7 or 8-14 in combination with the manual configuration illustrated in FIGS. 24-27.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A breathing circuit that delivers anesthetic gases to a patient, the breathing circuit comprising:
a hose having a proximal end coupled to an anesthetic gas delivery machine and a distal end;
a breathing apparatus attached to a patient to facilitate patient inhalation; and
a coupler having a proximal end connected to the distal end of the hose and a distal end connected to the breathing apparatus, wherein the coupler includes a body having an inner surface and an outer surface and a valve that allows gas from the anesthetic gas delivery machine to flow to the breathing apparatus in an opened position and to block gas from flowing from the anesthetic gas delivery machine to the breathing apparatus in a closed position.

2. The breathing circuit of claim 1, wherein the coupler automatically actuates the valve into the opened position when the breathing apparatus is connected to the coupler and places the valve into the closed position when the breathing apparatus is disconnected from the coupler.

3. The breathing circuit of claim 2, wherein the coupler comprises a plurality of leaflets having first ends and second ends, wherein the first ends of the leaflets are coupled to the valve, which is located internal to the inner surface of the body of the coupler, and the second ends of the leaflets radially extend from the outer surface of the body of the coupler, the plurality of leaflets actuate the valve both when the breathing apparatus is connected to the outer surface of the body of the coupler and when the breathing apparatus is connected to the inner surface of the body of the coupler.

4. The breathing circuit of claim 2, wherein the coupler comprises a plurality of legs having first ends and second ends, wherein the first ends of the plurality of legs are coupled to the valve, which is located internal to the inner surface of the body of the coupler, and the second ends are partially located external to the outer surface of the body of the coupler and partially located internal to the inner surface of the body of the coupler, wherein when the breathing apparatus is connected to the outer surface of the body of the coupler the plurality of legs actuate the valve by moving portions of the second ends that are located external to the outer surface of the body of the coupler to a position internal to the inner surface of the body of the coupler and wherein when the breathing apparatus is connected to the inner surface of the body of the coupler the plurality of legs actuate the valve by moving portions of the second ends that are located internal to the inner surface of the body of the coupler to a position external to the outer surface of the body.

5. The breathing circuit of claim 2, further comprising a spring having a proximal end and a distal end, wherein the proximal end of the spring is connected to a stop and the distal end of the spring is connected to a proximal end of the valve.

6. The breathing circuit of claim 5, further comprising a plurality of prongs having proximal ends and distal ends, wherein the proximal ends are coupled to the distal end of the valve and the distal ends of the plurality of prongs at least partially protrude through openings in the body of the coupler, wherein the plurality of prongs actuate the valve when the breathing apparatus is connected to the outer surface of the body of the coupler by pushing the distal ends of the plurality of prongs that partially protrude through the openings in the body against the valve and wherein the plurality of prongs actuate the valve when the breathing apparatus is connected to the inner surface of the body of the coupler by pushing the distal ends of the plurality of prongs that are located inside the coupler against the valve.

7. The breathing circuit of claim 1, wherein the valve is manually actuated into the opened position and manually actuated into the closed position.

8. The breathing circuit of claim 7, wherein valve comprises a sleeve that slides proximally and distally along an outer surface of the coupler and includes an inner facing channel.

9. The breathing circuit of claim 8, wherein the coupler further comprises:
a partition separating a distal region from a proximal region of the coupler and located internal to the inner surface of the body of the coupler;
a first set of holes that extend between the inner and outer surfaces of the body of the coupler and are located in the distal region of the coupler; and
a second set of holes that extend between the inner and outer surfaces of the body of the coupler and are located in the proximal region of the coupler;
wherein in the closed position the sleeve covers the second set of holes and leaves the first set of holes uncovered so the anesthetic gas is unable to flow from the proximal region into the distal region; and
wherein in the opened position the sleeve covers the first set of holes in the distal region and the second set of holes in the proximal region so the anesthetic gas can flow from the proximal region into the distal region via the inner facing channel in the sleeve.

10. A coupler that couples a hose from an anesthetic gas delivery machine to a breathing apparatus attached to a patient, the coupler comprising:
a body having an outer surface and an inner surface;
a proximal region that couples to a hose from an anesthetic gas delivery machine;
a distal region that couples to the breathing apparatus attached to the patient; and
a medial region located between the proximal region and the distal region;
a valve housed within the inner surface of the body of the coupler; and
wherein in an opened position the valve allows anesthetic gas to flow from the proximal region to the distal region and wherein in a closed position the valve blocks anesthetic gas from flowing from the proximal region to the distal region and from leaking into a surrounding environment.

11. The coupler of claim 10, wherein the valve is located at the distal end.

12. The coupler of claim 11, further comprising a plurality of leaflets having first ends and second ends, wherein the first ends of the leaflets are coupled to the valve and the second ends of the leaflets radially extend from the outer surface of the body of the coupler, the plurality of leaflets actuate the valve regardless of whether the breathing apparatus is connected to the outer surface of the body of the coupler in the distal region or connected to an inner surface of the body of the coupler in the distal region.

13. The coupler of claim 10, wherein the valve is located in the medial region.

14. The coupler of claim 13, further comprising a plurality of legs having first ends and second ends, wherein the first ends of the legs are coupled to the valve and the second ends of the legs are partially located outwardly from the outer surface of the body of the coupler and partially located inwardly from the inner surface of the body of the coupler, the plurality of legs actuate the valve regardless of whether the breathing apparatus is connected to the outer surface of the body of the coupler in the distal region and moves the portions of the second ends that are located outwardly from the outer surface of the body to a position inwardly from the inner surface of the body or whether the breathing apparatus is connected to the inner surface of the body of the coupler and moves the portions of the second ends that are located inwardly from the inner surface of the body to a position outwardly from the outer surface of the body of the coupler.

15. The coupler of claim 13, further comprising a spring located inside the coupler in the medial region and having a proximal end and a distal end, wherein the proximal end of the spring is connected to a stop and the distal end of the spring is connected to a proximal end of the valve.

16. The coupler of claim 15, further comprising a plurality of prongs having proximal ends and distal ends, wherein the proximal ends of the plurality of prongs are coupled to the distal end of the valve and the distal ends of the plurality of prongs at least partially protrude through openings in the body of the coupler in the distal region, the plurality of prongs actuate the valve when the breathing apparatus is connected to an outer surface of the body of the coupler by pushing the distal ends of the plurality of rigid prongs that partially protrude through the openings in the body against the valve and the plurality of prongs actuate the valve when the breathing apparatus is connected to an inner surface of the coupler by pushing the distal ends of the plurality of rigid prongs that are inside the body against the valve.

18. A coupler that couples a hose from an anesthetic gas delivery machine to a breathing apparatus attached to a patient, the coupler comprising:
a body having an outer surface and an inner surface;
a proximal region that couples to a hose from an anesthetic gas delivery machine;
a distal region that couples to the breathing apparatus attached to the patient; and
a medial region located between the proximal region and the distal region;
a valve accessible from the outer surface of the body of the coupler and manually actuated; and
wherein in an opened position the valve allows anesthetic gas to flow from the proximal region to the distal region and wherein in a closed position the valve blocks anesthetic gas from flowing from the proximal region to the distal region and from leaking into a surrounding environment.

19. The coupler of claim 16, wherein the valve comprises a sleeve having an inner facing channel that slides proximally and distally along an outer surface of the coupler.

20. The coupler of claim 17, further comprising:
a partition located inside the medial region of the coupler and separating the distal region from the proximal region;
a first set of holes that extend between the inner and outer surfaces of the body of the coupler and are located in the distal region of the coupler; and
a second set of holes that extend between the inner and outer surfaces of the body of the coupler and are located in the proximal region of the coupler;
wherein in the closed position the sleeve covers the second set of holes and leaves the first set of holes uncovered so the anesthetic gas is unable to flow from the proximal region into the distal region; and
wherein in the opened position the sleeve covers the first set of holes in the distal region and the second set of holes in the proximal region so the anesthetic gas can flow from the proximal region into the distal region via the inner facing channel in the sleeve.

21. A method of preventing anesthetic gases delivered to a patient by an anesthetic gas delivery machine from entering a surrounding environment, the method comprising:
attaching a proximal end of a coupler to a distal end of a hose that is coupled to an anesthetic gas delivery machine, the coupler including a valve that in a closed position;
turning on the anesthetic gas delivery machine so that anesthetic gases flow from the anesthetic gas delivery machine, through the hose and to the coupler, wherein the closed valve prevents anesthetic gas from entering into a surrounding environment; and
attaching a distal end of the coupler to a breathing apparatus that is attached to a patient, wherein attaching the distal end of the coupler to the breathing apparatus opens the valve so that anesthetic gas flows to the patient.

22. The method of claim 19, further comprising detaching the distal end of the coupler from the breathing apparatus, wherein detaching the distal end of the coupler from the breathing apparatus closes the valve to prevent anesthetic gas from entering the surrounding environment.
